(19) United States
(12) Patent Application Publication
(10) Pub. No.: US 2026/0355362 A1
(43) Pub. Date: Nov. 12, 2026

(54) MIXTURE, KIT, PREPARATION METHOD, AND USE FOR PREPARING PHARMACEUTICAL COMPOSITION FOR DIAGNOSING AND TREATING CARDIOVASCULAR DISEASE

(71) Applicant: National Atomic Research Institute, Taoyuan (TW)

(72) Inventors: SU-JUNG CHEN, Taoyuan (TW); CHUN-TANG CHEN, Taoyuan (TW); MING-WEI CHEN, Taoyuan (TW); YA-JEN CHANG, Taoyuan (TW); MAO-CHI WENG, Taoyuan (TW); CHIEN-CHUNG HSIA, Taoyuan (TW); CHENG-LIANG PENG, Taoyuan (TW); WEI-HSI CHEN, Taoyuan (TW); SIAO-SYUN GUAN, Taoyuan (TW)

(21) Appl. No.: 19/259,114

(22) Filed: Jul. 3, 2025

(30) Foreign Application Priority Data
May 7, 2025 (TW) 114117135

(51) Int. Cl. A61K 51/04

(52) U.S. Cl. CPC A61K 51/0497; A61K 2123/00

(57) ABSTRACT

Provided herein are a mixture, a kit, a preparation method, and use for preparing a pharmaceutical composition for diagnosing and treating a cardiovascular disease. The mixture includes an APD compound or a salt thereof, and a 0.5-1 M acetate-based buffer, and may further include a stabilizer as required. Specifically, a weight ratio of the APD compound or the salt thereof to the stabilizer is in a range of 1:0.5 to 1:5, and the mixture has a pH value in a range of 4.5 to 6.

### CROSS REFERENCE TO RELATED APPLICATION

The present invention claims priority to the Taiwan Patent Application Serial No. 114117135, filed on May 7, 2025, the disclosure of which is incorporated by references herein in its entirety.

### BACKGROUND

### Technical Field

The present invention relates to the field of medical imaging, and in particular, to a mixture for diagnosing and treating a cardiovascular disease and for rapid labeling of a radioisotope, a kit, and a preparation method.

### Related Art

Atherosclerosis caused by vascular inflammation is one of the primary pathological mechanisms of modern cardiovascular diseases, and is mainly characterized by, at early stages, “silent inflammation”, “immune cell infiltration”, “endothelial cell activation”, and other processes. However, at these stages, plaques have not yet calcified or caused significant stenosis, making it difficult to detect the plaques by conventional clinical examination methods.

Currently, common radiopharmaceuticals used in nuclear medicine imaging for atherosclerosis diagnosis include: F-18-NaF (reflecting vascular calcification), F-18-FDG (assessing local glucose metabolism), and T1-201 (assessing myocardial perfusion). These radiopharmaceuticals primarily focus on the detection of mid to late-stage plaques, but are not sensitive enough for early inflammation or immune-related pathological changes.

The chemokine receptor CXCR4 has been shown to be significantly expressed at the early stage of atherosclerosis, especially in macrophages and inflammatory T-cell populations, and has therefore become an important target for the development of early cardiovascular imaging biomarkers in recent years. Clinically, although radiopharmaceuticals targeting CXCR4, for example, Ga-68-Pentixafor, have been developed and used in positron emission tomography/computed tomography (PET/CT) imaging studies, there are still many limitations. For example, a reagent vial needs to undergo lyophilization and cryogenic storage, resulting in a cumbersome preparation process and high costs.

In view of this, there is an urgent need in this field to provide an easy-to-prepare pharmaceutical composition for diagnosing and treating CXCR4-related diseases, to overcome the shortcomings in the related art.

### SUMMARY

For readers to understand the basic meaning of the present disclosure, the summary provides a brief description of the present disclosure. The summary is not a complete description of the present disclosure and is not intended to define the technical features or scope of rights of the present invention.

To overcome the shortcomings in the related art, a first aspect of the present invention provides a mixture for rapid labeling of a radioisotope. The mixture includes an APD (((6-aminomethyl)-pyridin-2-yl-methyl)-pyridin-2-yl-methyl-[3-(1,4,7,10-tetraaza-cyclododec-1-yl)-propyl]-amine) compound or a salt thereof capable of binding CXCR4, a 0.5-1 M acetate-based buffer, and a stabilizer. A weight ratio of the APD compound or the salt thereof to the stabilizer is in a range of 1:0.5 to 1:5, and a pH value of the mixture is in a range of 4.5 to 6.5.

Further, in a specific implementation, the stabilizer is potassium sodium tartrate, disodium tartrate, or a combination thereof.

Another aspect of the present invention provides an assay kit for rapid labeling of a radioisotope, including a container and an instruction for use. The container contains the mixture according to any one of the foregoing implementations, for the addition of a metal radioisotope. In a non-limiting implementation, the metal radioisotope is Ga-67, Ga-68, Cu-64, Cu-67, Ac-225, In-111, Tc-99m, Y-90, or the like.

In another implementation, the instruction for use in the assay kit of the present invention is used to guide a user to prepare, by using the kit of the present invention, an APD drug labeled with a radioisotope.

Still another aspect of the present invention provides a method for rapidly preparing a contrast agent for detecting atherosclerosis, including the following steps: providing the mixture according to any one of the foregoing implementations; and providing a solution containing a metal radioisotope Ga-67, Ga-68, Cu-64, Cu-67, Ac-225, In-111, Tc-99m, or Y-90, and adding the solution containing the metal radioisotope to the mixture to carry out a reaction at 35-100 °C for 10-30 min, to obtain a contrast agent.

In a specific implementation, the solution containing the metal radioisotope is a Ga-68 or Ga-67 solution with an activity of 5-30 mCi, and an appropriate labeling condition is that the solution containing the metal radioisotope and the mixture react at 80-100 °C for at least 15 min.

According to another implementation of the present invention, the solution containing the metal radioisotope is a Cu-64 or Cu-67 solution with an activity of 1-100 mCi, and an appropriate labeling condition is that the solution containing the metal radioisotope and the mixture react at 35-65 °C for at least 15 min.

In addition, still another aspect of the present invention provides use of the mixture according to any one of the foregoing implementations for preparing a pharmaceutical composition for diagnosing and treating atherosclerosis.

A person of ordinary skill in the technical field to which the present invention belongs can fully understand the central concept, adopted technical means, and various implementations of the present invention with reference to the following implementations.

### BRIEF DESCRIPTION OF THE DRAWINGS

To make the foregoing descriptions of the present invention and other objectives, features, advantages, and embodiments more apparent and understandable, the drawings are described as follows:

FIG. 1A and FIG. 1B show results of positron emission tomography/computed tomography (PET/CT) of ApoE^{-/-} mice with a Ga-68-APD drug injected. FIG. 1A shows that the drug injected into the mice accumulates in the heart, kidney, and bladder. FIG. 1B shows results of ex vivo PET imaging, where signals accumulate in a lesion site (near the aortic arch) of the ApoE^{-/-} mouse.

### DETAILED DESCRIPTION

To make the description of the present disclosure more exhaustive and complete, the following provides illustrative textual descriptions for the implementations and specific embodiments of the present invention, but the implementations and specific embodiments of the present invention are not limited thereto.

Unless otherwise indicated, scientific and technical terms used in this specification have the same meaning as understood and commonly used by a person of ordinary skill in the art. Further, a noun used in this specification covers both the singular and plural forms of the noun, unless otherwise indicated.

The word “individual” or “patient” refers to an animal capable of accepting the mixture and kit of the present invention, and the pharmaceutical composition prepared by using the method according to the present invention. In a preferred implementation, the animal is a mammal, and in particular, a human.

As described in this specification, the word “about” usually means that an actual value is plus or minus 10%, 5%, 1%, or 0.5% of a specific value or range. The word “about” herein means that an actual value falls within an acceptable standard error of a mean, depending on the consideration of a person of ordinary skill in the technical field to which the present invention belongs. With the exception of examples, or unless otherwise expressly stated, it is to be understood that the ranges, quantities, values, and percentages herein are modified by the word “about”. Accordingly, unless otherwise indicated, the values or parameters disclosed in this specification and in the appended claims are all approximate values and may be changed as required.

To overcome the shortcomings in the related art, the present invention provides a mixture and kit for rapid labeling of a radioisotope, and use of the mixture for preparing a pharmaceutical composition for diagnosing and treating a cardiovascular disease. The technical content disclosed in the present invention has the following advantages: 1. The labeling condition is simple (short heating duration), storage can be carried out at room temperature, and a solution vial can be sterilized at high temperature. 2. Radioisotope labeling can be instantly carried out at clinical sites, improving application efficiency and suitability for diagnostic and/or therapeutic use of short-half-life nuclides. Therefore, the technical content disclosed in the present invention provides a new option for early molecular imaging diagnosis and/or treatment of a cardiovascular disease, especially vascular inflammation and atherosclerosis.

The present invention discloses a mixture capable of being rapidly radiolabeled. The mixture includes an APD compound or a salt thereof and a 0.5-1 M acetate-based buffer, and the mixture has a pH value in a range of 4.5 to 6.

In another implementation of the present invention, the mixture of the present invention further includes a stabilizer, where a weight ratio of the APD compound or the salt thereof to the stabilizer is in a range of 1:0.5 to 1:5. For example, the weight ratio of the APD compound or the salt thereof to the stabilizer is 1:0.5, 1:0.6, 1:0.7, 1:0.8, 1:0.9, 1:1, 1:1.5, 1:2, 1:2.5, 1:3, 1:3.5, 1:4, 1:4.5, or 1:5.

Further, the stabilizer of the present invention is potassium sodium tartrate, disodium tartrate, or a combination thereof.

Another aspect of the present invention provides an assay kit for rapid radiolabeling, including a container and an instruction for use. The container contains the mixture according to any one of the foregoing implementations. In a non-limiting implementation, the assay kit of the present invention further includes a metal radioisotope, including but not limited to Ga-67, Ga-68, Cu-64, Cu-67, Ac-225, In-111, Tc-99m, or Y-90. It is noted that the metal radioisotope may be produced by using an accelerator or a reactor. The kit disclosed in the present invention is convenient for clinical use, making the preparation of an APD drug with a radioisotope easier and more convenient. The kit is suitable to be promoted for use in the clinical field.

Still another aspect of the present invention provides a method for rapidly preparing a contrast agent for detecting atherosclerosis. In this method, the mixture of the present invention and a metal radioisotope react at 35-100 °C for 10-30 min, to rapidly obtain a contrast agent.

In an implementation, the metal radioisotope is Ga-68 or Ga-67 with an activity of 5-30 mCi, and an appropriate labeling condition is that the reaction is carried out at 80-100 °C for at least 15 min. In an embodiment of the present invention, duration for the reaction is about 15-30 min.

According to another implementation of the present invention, the metal radioisotope is Cu-64 or Cu-67 with an activity of 1-100 mCi, and an appropriate labeling condition is that the reaction is carried out at 35-65 °C for at least 15 min. In an embodiment of the present invention, duration for the reaction is about 15-30 min. A person of ordinary skill in the technical field to which the present invention belongs should understand that the activity may be selected according to needs of practical use. For example, the mixture of the present invention labeled with Cu-64 may be used as a diagnostic agent for PET, with an activity of about 5-10 mCi; and the mixture of the present invention labeled with Cu-64 may be used as a therapeutic agent, with an activity of about 30-60 mCi.

In addition, the APD drug prepared by using the mixture of the present invention with a metal radioisotope can effectively accumulate in an inflamed region of the heart, and therefore has the potential to be used as a pharmaceutical composition for diagnosing and treating a cardiovascular disease, especially vascular inflammation and atherosclerosis. Therefore, still another aspect of the present invention provides use of the mixture according to any one of the foregoing implementations for preparing a pharmaceutical composition for diagnosing and treating a cardiovascular disease.

A plurality of examples is disclosed as follows to describe various different implementations of the present invention, so that a person of ordinary skill in the technical field to which the present invention belongs can implement the technical content disclosed in the present invention according to the disclosure of this specification. Therefore, the examples disclosed as follows cannot be used to limit the scope of rights of the present invention.

### Example 1: Preparation of the Mixture of the Present Invention

### 1.1 Mixture A of the Present Invention

To a container was added 1.5-3 mL of the mixture of the present invention containing 300-500 μg of the APD compound or the salt thereof, a 0.5-1 M sodium acetate buffer, and 300-600 μg of the stabilizer. The stabilizer is potassium sodium tartrate or disodium tartrate, and a pH value of the mixture of the present invention is in a range of 4.5 to 6. The specific composition of the mixture A of the present invention is shown in Table 1.

TABLE 1

| Number | Composition of the mixture A | Radioisotope labeling effect |
|---|---|---|
| 1 | 300 μg of APD•6HCl; 400 μg of | Ga-68 activity 7-10 mCi, volume 0.5 |
| | potassium sodium tartrate | mL, both purity and labeling |
| | 0.5M sodium acetate buffer | efficiency greater than 95% |
| 2 | 440 μg of APD•6HCl; 600 μg of | Ga-68 activity 15-30 mCi, volume |
| | potassium sodium tartrate | 1.5 mL, |
| | 0.5M sodium acetate buffer | both purity and labeling efficiency |
| | (solution vial) | greater than 95% |
| 3 | 440 μg of APD•6HCl; 480 μg of | Measured Ga-68 activity 21 mCi and 7 |
| | disodium tartrate | mCi, volume 2 mL, both radiochemical |
| | 0.75M sodium acetate buffer | purity and labeling efficiency |
| | (solution vial) | greater than 95% |

### 1.2 Mixture B of the Present Invention

To a container was added 1.5-3 mL of the mixture of the present invention containing 200-2000 μg of the APD compound or the salt thereof and a 0.5-1 M sodium acetate buffer. A pH value of the mixture of the present invention is in a range of 4.5 to 6. The optimal condition is 2000 μg of APD·6HCl. A solution vial containing a 0.5 M sodium acetate buffer (pH 5.6) may be used for labeling with Cu-64 of 2 mCi. The labeling efficiency and the radiochemical purity may be greater than 90%.

### 1.3 Mixture C of the Present Invention

An SCX column was activated with a mixed solution of 5 M NaCl/0.1 N HCl and activated with deionized water. Then, 5 mL of radioisotope Ga-68 solution of 5-30 mCi was loaded onto the SCX column for adsorption and then pushed out until dry. Then, 1 mL of mixed solution of 5 M NaCl/0.1 N HCl was taken and injected into an upper end of the SCX column to elute out the isotope Ga-68 in a final concentrated volume of 1 mL. The concentrated Ga-68 is used to label the APD mixture A, and in this case, both labeling efficiency and radiochemical purity can be greater than 90%.

In this way, the volume of a drug for radiodiagnosis and radiotherapy can be greatly reduced, so that the labeling step can be completed in a vial, to quickly and conveniently prepare a contrast agent for diagnosing and treating a cardiovascular disease.

### Example 2: Rapid Preparation of a Contrast Agent for Diagnosing and Treating Vascular Inflammation and Atherosclerosis by Using the Mixture of the Present Invention

In this example, an APD drug labeled with a metal radioisotope was prepared according to a method for rapid labeling of the metal radioisotope Ga-68 or Ga-67 on the mixture A in Example 1.1 of the present invention. The APD drug can be used as a contrast agent for detecting vascular inflammation and atherosclerosis. 0.5-2.5 mL of metal radioisotope solution (for example, Ga-68 or Ga-67 with an activity of 7-30 mCi) was added to the mixture A of the present invention. In a non-limiting implementation, the metal radioisotope solution is prepared by using an accelerator or a reactor. Then, the mixture containing the metal radioisotope was heated to 80-100 °C to react for 15 min, to obtain an APD compound or a salt thereof labeled with the metal radioisotope Ga-68 or Ga-67. The labeling efficiency was verified by radio thin layer chromatography (ITLC), and the radiochemical purity was verified by radio high-pressure liquid chromatography (HPLC). The results show that the labeling efficiency and the chemical purity are 96.5% and 94.6% respectively, with a single radioactive peak, indicating that the reaction is stable. In an embodiment, a Ga-68-labeled APD drug in the present invention can remain stable in a solution of saline and fetal bovine serum (FBS) (1:5) for at least 1 h. The results are shown in Table 2. The results show that the Ga-68-labeled APD drug in the present invention is suitable for clinical diagnostic and therapeutic use.

TABLE 2

TLC analysis

| | | Ga-68 APD | Ga-68 APD | |
|---|---|---|---|---|
| | | at room | in FBS | HPLC analysis |
| | | temperature | (1:5) | Ga-68 APD |
| 0 | h | 96.5% | 96.0% | 94.6% |
| 0.5 | h | 96.6% | 92.5% | 91.9% |
| 1 | h | 100% | 90.1% | 92.6% |
| 1.5 | h | 96% | 90.6% | 90.1% |
| 2 | h | 96% | 84.6% | 90.7% |

### Example 3: Preparation of an APD Drug Solution Vial of the Present Invention

### 3.1 Preparation of an APD Drug Solution Vial a of the Present Invention

In this example, to a container was added 1.5-3 mL of the mixture of the present invention containing 300-500 μg of the APD compound or the salt thereof, a 0.5-1 M sodium acetate buffer, and 300-500 μg of the stabilizer. The stabilizer is potassium sodium tartrate or disodium tartrate, and a pH value of the mixture of the present invention is in a range of 4.5 to 6. After preparation, the container was sterilized at 121 °C under high pressure for 1 h to obtain the APD drug solution vial A. The APD drug solution vial A was stored in the dark at room temperature for use.

### 3.2 Preparation of an APD Drug Solution Vial B of the Present Invention

In this example, to a container was added 1.5-3 mL of the mixture of the present invention containing 300-500 μg of the APD compound or the salt thereof and a 0.5-1 M sodium acetate buffer. A pH value of the mixture of the present invention is in a range of 4.5 to 6. After preparation, the container was sterilized at 121 °C under high pressure for 1 h to obtain the APD drug solution vial B. The APD drug solution vial B was stored in the dark at room temperature for use.

### Example 4: Stability Test of the APD Drug Solution Vial of the Present Invention

### 4.1 Stability Test of the APD Drug Solution Vial A

The APD drug solution vial A was placed at room temperature to visually observe changes in appearance, and the labeling efficiency was determined periodically according to the method in Example 2 after labeling with a Ga-68 or Ga-67 radioisotope solution. The labeling efficiency is higher than 90%. The results are shown in Table 3.

TABLE 3

| | |
|---|---|
| Storage duration | Ga-68-labeled APD solution vial |

| 4 weeks | (RT) | (−20 °C) | 4 °C |
|---|---|---|---|
| Radiochemical purity | 95.6% | 95.1% | 94.4% |
| Labeling efficiency | 94.3% | 93.3% | 94.2% |

Through at least four weeks of observation, the APD drug solution vial A has no clear changes in appearance, and the Ga-68 or Ga-67 radioisotope labeling efficiency is still greater than 90%, indicating that the solution vial has good storage stability after high-temperature and high-pressure sterilization. Further, through continuous observation, the sterilized APD drug solution vial of the present invention is still stable for at least three months.

The foregoing results show that the sterilizable APD drug solution vial can be conveniently prepared and can be easily used for labeling of the short-half-life radioisotope Ga-68, Ga-67, Cu-64, or Cu-67, and the APD structure is stable for storage and preparation compared with a peptide compound that is prone to hydrolysis. Further, the sterilized APD drug solution vial of the present invention is still stable for at least three months.

### Example 5: The Ga-68-Labeled APD Drug of the Present Invention can be Used for Diagnosing and Treating Atherosclerosis

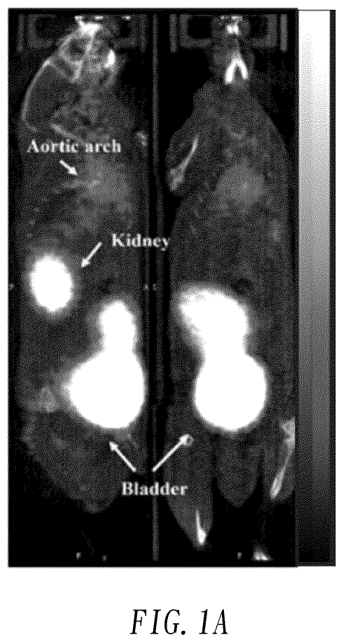

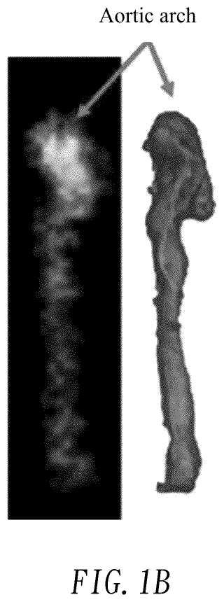

ApoE^{-/-} mice were subjected to positron emission tomography/computed tomography (PET/CT) imaging. At the beginning of the test, the Ga-68-labeled APD drug of Example 1 was administered by tail vein injection (0.2-0.3 mCi/0.1 mL for each mouse) (n=5). After injection, the mice were anesthetized with 1.5% isoflurane. The anesthetized mice were subjected to Mediso nanoPET/CT imaging for 1 h, and one image was captured every half hour. The original images were reconstructed and analyzed using VivaQuant software. As shown in FIG. 1A and FIG. 1B, the imaging results show that the Ga-68-labeled APD drug injected into the mice accumulates in the heart, kidney, and bladder. The results are similar to in-vivo distribution research results. It can be learned that the Ga-68-labeled APD drug of the present invention has the potential to be used as a pharmaceutical composition for diagnosing and treating vascular inflammation or atherosclerosis.

The specific examples disclosed above are not intended to limit the scope of rights of the present invention. A person of ordinary skill in the art may make modifications in accordance with common experience without departing from the principle and spirit of the present invention. Therefore, the scope of rights of the present invention is subject to the scope of the claims.

What is claimed is:

1. A mixture for rapid labeling of a radioisotope, comprising:
an APD compound or a salt thereof; and
a 0.5-1 M acetate-based buffer, wherein the mixture has a pH value in a range of 4.5 to 6.5.

2. The mixture according to claim 1, further comprising a stabilizer, wherein a weight ratio of the APD compound or the salt thereof to the stabilizer is in a range of 1:0.5 to 1:5.

3. The mixture according to claim 2, wherein the stabilizer is potassium sodium tartrate, disodium tartrate, or a combination thereof.

4. An assay kit for rapid labeling of a radioisotope, comprising:
a container, containing the mixture according to claim 1; and
an instruction for use.

5. The assay kit according to claim 4, further comprising a metal radioisotope, wherein the metal radioisotope is Ga-67, Ga-68, Cu-64, Cu-67, Ac-225, In-111, Tc-99m, or Y-90.

6. A method for rapidly preparing a contrast agent for detecting a cardiovascular disease, comprising:
(a) providing the mixture according to claim 1; and
(b) providing a solution containing a metal radioisotope, and adding the solution containing the metal radioisotope to the mixture to carry out a reaction at 35-100 °C for 10-30 min, to obtain a contrast agent.

7. The method according to claim 6, wherein the metal radioisotope in the solution containing the metal radioisotope is Ga-67, Ga-68, Cu-64, Cu-67, Ac-225, In-111, Tc-99m, or Y-90.

8. The method according to claim 7, wherein the mixture further comprises a stabilizer, wherein a weight ratio of the APD compound or the salt thereof to the stabilizer is in a range of 1:0.5 to 1:5, and the metal radioisotope is Ga-67 or Ga-68.

9. The method according to claim 8, wherein the metal radioisotope in the solution containing the metal radioisotope is Ga-67 or Ga-68 with an activity of 5-30 mCi.

10. The method according to claim 9, wherein in step (b), the reaction is carried out at 80-100 °C for at least 15 min.

11. The method according to claim 7, wherein the metal radioisotope in the solution containing the metal radioisotope is Cu-64 or Cu-67 with an activity of 1-100 mCi.

12. The method according to claim 11, wherein in step (b), the reaction is carried out at 35-65 °C for at least 15 min.

13. Use of the mixture according to claim 1 for preparing a pharmaceutical composition for diagnosing and treating a cardiovascular disease.
